(19) United States
(12) Patent Application Publication
(10) Pub. No.: US 2024/0238056 A1
(43) Pub. Date: Jul. 18, 2024

(54) CONTROL SYSTEM, OPERATION DEVICE, CONTROL METHOD, AND COMPUTER READABLE MEDIUM

(71) Applicant: TOYOTA JIDOSHA KABUSHIKI KAISHA, Toyota-shi Aichi-ken (JP)

(72) Inventors: Yukinori KURAHASHI, Toyota-shi Aichi-ken (JP); Shiro ODA, Anjo-shi Aichi-ken (JP); Takeshi MATSUI, Nissin-shi Aichi-ken (JP)

(73) Assignee: TOYOTA JIDOSHA KABUSHIKI KAISHA, Toyota-shi Aichi-ken (JP)

(21) Appl. No.: 18/410,503

(22) Filed: Jan. 11, 2024

(30) Foreign Application Priority Data
Jan. 17, 2023 (JP) 2023-004930

(51) Int. Cl. A61B 34/32; A61B 34/00; A61B 34/37

(52) U.S. Cl. CPC A61B 34/32; A61B 34/37; A61B 34/74; A61B 2034/742; A61B 2560/0437; A61B 2560/0487

(57) ABSTRACT

A control system according to the present disclosure includes an operation device and a control unit. The operation device includes a movement operation unit configured to accept a moving operation for moving a mobile robot and a switching operation unit configured to accept an operation for switching the modes between the autonomous moving mode and the user operation mode. The control unit switches whether or not to accept the moving operation by the movement operation unit based on information indicating which one of the autonomous moving mode and the user operation mode the mobile robot is in and a change in a switching signal in response to the switching operation.

### CROSS REFERENCE TO RELATED APPLICATIONS

This application is based upon and claims the benefit of priority from Japanese patent application No. 2023-4930, filed on Jan. 17, 2023, the disclosure of which is incorporated herein in its entirety by reference.

### BACKGROUND

The present disclosure relates to a control system, an operation device, a control method, and a program.

Japanese Patent No. 6171541 discloses an autonomous mobile body capable of switching its mode between an autonomous traveling mode and a manual traveling mode.

### SUMMARY

The applicant has studied a case in which a user operates, using an operation device, a mobile robot capable of switching between an autonomous moving mode and a user operation mode. When a user performs an unnecessary operation input when he/she performs an operation of switching the above modes using the operation device, the mobile robot may perform an unintended movement due to the unnecessary input by the user.

The present disclosure has been made in order to solve the aforementioned problem, and provides a control system, an operation device, a control method, and a program capable of preventing a mobile robot capable of switching between an autonomous moving mode and a user operation mode by a user performing an operation of switching these modes using an operation device from performing an unintended movement due to an unnecessary operation input that may occur when the user operates this operation device.

A control system according to the present disclosure is a control system for controlling a mobile robot capable of switching between an autonomous moving mode in which the mobile robot autonomously moves and a user operation mode in which the mobile robot moves based on a user operation, the control system including: an operation device including a movement operation unit configured to accept a moving operation for moving the mobile robot and a switching operation unit configured to accept a switching operation for switching the modes between the autonomous moving mode and the user operation mode; and a control unit configured to switch, based on information indicating which one of the autonomous moving mode and the user operation mode the mobile robot is in and a change in a switching signal in response to the switching operation, whether or not to accept the moving operation by the movement operation unit. Since this control system performs the aforementioned configuration, it is possible to prevent a mobile robot from performing an unintended movement due to an unnecessary operation input that may occur when a user performs an operation of switching modes using an operation device.

When the mobile robot is in the autonomous moving mode, the control unit may switch the mode to the user operation mode when falling of the switching signal in response to the switching operation has been detected. The control system switches the modes as described above, whereby it is possible to prevent an unintended movement of the mobile robot due to an unnecessary input at the time of switching from the autonomous moving mode to the user operation mode.

When the mobile robot is in the autonomous moving mode, the control unit may stop or decelerate the mobile robot when falling of the switching signal in response to the switching operation has been detected, and switch the mode to the user operation mode after a predetermined period of time has elapsed. The control system switches the modes as described above, whereby it is possible to prevent an unintended movement of the mobile robot due to an unnecessary input at the time of switching from the autonomous moving mode to the user operation mode for a longer period of time.

When the mobile robot is in the user operation mode, the control unit may switch the mode to the autonomous moving mode when rising of the switching signal in response to the switching operation has been detected. The control system switches the modes as described above, whereby it is possible to prevent an unintended movement of the mobile robot due to an unnecessary input at the time of switching from the user operation mode to the autonomous moving mode.

The operation device may be a joystick device including a stick member and a button disposed in the stick member, the stick member being the movement operation unit and the button being the switching operation unit. The control system performs the configuration as described above, whereby it is possible to prevent an unintended movement of the mobile robot due to an unnecessary operation input at a time of operation of switching the modes also in the joystick device with which a moving operation can be intuitively performed.

An operation device according to the present disclosure is an operation device configured to operate a mobile robot capable of switching between an autonomous moving mode in which the mobile robot autonomously moves and a user operation mode in which the mobile robot moves based on a user operation, the operation device including: a movement operation unit configured to accept a moving operation for moving the mobile robot; a switching operation unit configured to accept a switching operation for switching the modes between the autonomous moving mode and the user operation mode; a reception unit configured to receive a signal indicating whether the mobile robot is in the autonomous moving mode or the user operation mode; and an output unit configured to output a mode switching signal to the mobile robot to switch whether or not to accept the moving operation by the movement operation unit based on the signal received by the reception unit and a change in a switching signal in response to the switching operation. The operation device includes the aforementioned configuration, whereby it is possible to prevent a mobile robot from performing an unintended movement due to an unnecessary operation input that may occur when a user performs an operation of switching modes using an operation device.

When the mobile robot is in the autonomous moving mode, the output unit may output a signal for switching the mode to the user operation mode to the mobile robot as the mode switching signal when falling of the switching signal in response to the switching operation has been detected. With the aforementioned configuration, it is possible to prevent an unintended movement of the mobile robot due to an unnecessary input at the time of switching from the autonomous moving mode to the user operation mode.

When the mobile robot is in the autonomous moving mode, the output unit may output a control signal for stopping or decelerating the mobile robot when falling of the switching signal in response to the switching operation has been detected, and output a signal for switching the mode to the user operation mode to the mobile robot as the mode switching signal after a predetermined period of time has elapsed. The operation device switches the modes as described above, whereby it is possible to prevent an unintended movement of the mobile robot due to an unnecessary input at the time of switching from the autonomous moving mode to the user operation mode for a longer period of time.

When the mobile robot is in the user operation mode, the output unit may output a signal for switching the mode to the autonomous moving mode to the mobile robot as the mode switching signal when rising of the switching signal in response to the switching operation has been detected. With the aforementioned configuration, it is possible to prevent an unintended movement of the mobile robot due to an unnecessary input at the time of switching from the user operation mode to the autonomous moving mode.

The operation device may be a joystick device including a stick member and a button disposed in the stick member, the stick member being the movement operation unit and the button being the switching operation unit. With the aforementioned configuration, it is possible to prevent an unintended movement of the mobile robot due to an unnecessary operation input at a time of operation of switching the modes also in a case where a joystick device with which a moving operation can be intuitively performed is used.

A control method according to the present disclosure is a control method for controlling a mobile robot capable of switching between an autonomous moving mode in which the mobile robot autonomously moves and a user operation mode in which the mobile robot moves based on a user operation using an operation device, in which the operation device includes a movement operation unit configured to accept a moving operation for moving the mobile robot and a switching operation unit configured to accept a switching operation for switching the modes between the autonomous moving mode and the user operation mode, and the control method switches whether or not to accept the moving operation by the movement operation unit based on information indicating which one of the autonomous moving mode and the user operation mode the mobile robot is in and a change in a switching signal in response to the switching operation. The control method includes the aforementioned configuration, whereby it is possible to prevent a mobile robot from performing an unintended movement due to an unnecessary operation input that may occur when a user performs an operation of switching modes using an operation device.

When the mobile robot is in the autonomous moving mode, the mode may be switched to the user operation mode when falling of the switching signal in response to the switching operation has been detected. The control method performs the aforementioned switching, whereby it is possible to prevent an unintended movement of the mobile robot due to an unnecessary input at the time of switching from the autonomous moving mode to the user operation mode.

When the mobile robot is in the autonomous moving mode, the mobile robot may be stopped or decelerated when falling of the switching signal in response to the switching operation has been detected, and the mode may be switched to the user operation mode after a predetermined period of time has elapsed. The control method performs the aforementioned switching, whereby it is possible to prevent an unintended movement of the mobile robot due to an unnecessary input at the time of switching from the autonomous moving mode to the user operation mode for a longer period of time.

When the mobile robot is in the user operation mode, the mode may be switched to the autonomous moving mode when rising of the switching signal in response to the switching operation has been detected. The control method performs the aforementioned switching, whereby it is possible to prevent an unintended movement of the mobile robot due to an unnecessary input at the time of switching from the user operation mode to the autonomous moving mode.

The operation device may be a joystick device including a stick member and a button disposed in the stick member, the stick member being the movement operation unit and the button being the switching operation unit. The control method may prevent an unintended movement of the mobile robot due to an unnecessary operation input at a time of operation of switching the modes also in a case where a joystick device with which a moving operation can be intuitively performed is employed as the operation device.

A program according to the present disclosure is a program for controlling a mobile robot capable of switching between an autonomous moving mode in which the mobile robot autonomously moves and a user operation mode in which the mobile robot moves based on a user operation using an operation device, in which the operation device includes a movement operation unit configured to accept a moving operation for moving the mobile robot and a switching operation unit configured to accept a switching operation for switching the modes between the autonomous moving mode and the user operation mode, the program is a program for causing a control computer included in the mobile robot or connected to the mobile robot to execute mode switching processing, and the mode switching processing is processing for switching whether or not to accept the moving operation by the movement operation unit based on information indicating which one of the autonomous moving mode and the user operation mode the mobile robot is in and a change in a switching signal in response to the switching operation. The program includes the aforementioned configuration, whereby it is possible to prevent a mobile robot from performing an unintended movement due to an unnecessary operation input that may occur when a user performs an operation of switching modes using an operation device.

The mode switching processing may include processing for switching, when the mobile robot is in the autonomous moving mode, the mode to the user operation mode when falling of the switching signal in response to the switching operation has been detected. The program switches the modes as described above, whereby it is possible to prevent an unintended movement of the mobile robot due to an unnecessary input at the time of switching from the autonomous moving mode to the user operation mode.

The mode switching processing may include processing for stopping or decelerating, when the mobile robot is in the autonomous moving mode, the mobile robot when falling of the switching signal in response to the switching operation has been detected, and switching the mode to the user operation mode after a predetermined period of time has elapsed. The program switches the modes as described above, whereby it is possible to prevent an unintended movement of the mobile robot due to an unnecessary input at the time of switching from the autonomous moving mode to the user operation mode for a longer period of time.

The mode switching processing may include processing for switching, when the mobile robot is in the user operation mode, the mode to the autonomous moving mode when rising of the switching signal in response to the switching operation has been detected. The program switches the modes as described above, whereby it is possible to prevent an unintended movement of the mobile robot due to an unnecessary input at the time of switching from the user operation mode to the autonomous moving mode.

The operation device may be a joystick device including a stick member and a button disposed in the stick member, the stick member being the movement operation unit and the button being the switching operation unit. The program may prevent an unintended movement of the mobile robot due to an unnecessary operation input at a time of operation of switching the modes also in a case where a joystick device with which a moving operation can be intuitively performed is employed as the operation device.

According to the present disclosure, it is possible to provide a control system, an operation device, a control method, and a program capable of preventing a mobile robot capable of switching between an autonomous moving mode and a user operation mode by a user performing an operation of switching these modes using an operation device from performing an unintended movement due to an unnecessary operation input that may occur when the user operates this operation device.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a schematic perspective view showing one example of a mobile robot as a control system according to an embodiment;

FIG. 2 is a top view showing one example of a joystick device mounted on the mobile robot shown in FIG. 1;

FIG. 3 is a timing chart showing an example of response to operations by a button and a stick member in a mobile robot according to a first comparative example;

FIG. 4 is a timing chart showing an example of response to operations by a button and a stick member in a mobile robot according to a second comparative example;

FIG. 5 is a timing chart showing one example of response to operations by a button and a stick member in the mobile robot shown in FIG. 1;

FIG. 6 is a flowchart for describing one example of a control method in the mobile robot shown in FIG. 1;

FIG. 7 is a timing chart showing another example of response to operations by a button and a stick member in the mobile robot shown in FIG. 1;

FIG. 8 is a flowchart for describing another example of the control method in the mobile robot shown in FIG. 1;

FIG. 9 is a schematic diagram showing one configuration example of a conveyance system including a control system according to the embodiment;

FIG. 10 is a flowchart for describing one example of a control method of the mobile robot in the conveyance system shown in FIG. 9;

FIG. 11 is a perspective view showing one example of a joystick device that operates a mobile robot included in a control system according to an alternative example;

FIG. 12 is a top view showing one example of an operation device that operates the mobile robot included in the control system according to the alternative example;

FIG. 13 is a top view showing another example of the operation device that operates the mobile robot included in the control system according to the alternative example; and

FIG. 14 is a diagram showing one example of a hardware configuration of a device.

### DESCRIPTION OF EMBODIMENTS

The present disclosure will be described hereinafter through embodiments of the disclosure, but the present disclosure according to the claims is not limited to the below-shown embodiments. Further, all the components/structures described in an embodiment are not necessarily for solving the problem.

### Embodiments

A control system according to this embodiment, which is a system that controls a mobile robot capable of switching between an autonomous moving mode in which the mobile robot autonomously moves and a user operation mode in which the mobile robot moves based on a user operation, includes an operation device and a control unit that switches these modes. Hereinafter, a joystick device will be described as an example of the operation device. Note that the user operation mode may also be referred to as a manual mode.

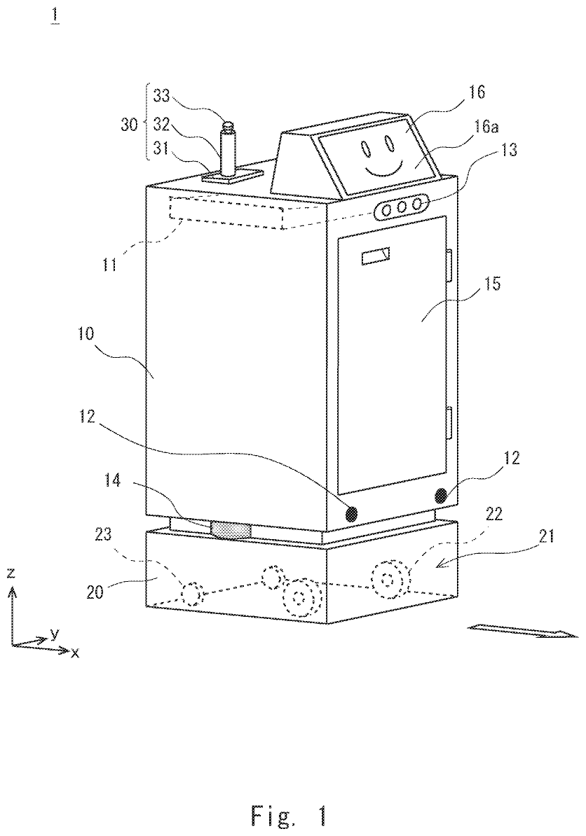

The functions of the aforementioned control unit may be disposed in a plurality of apparatuses in the aforementioned control system in a distributed manner, and this joystick device may be separated from the mobile robot. First, with reference to FIGS. 1 to 8, a configuration example in which the aforementioned joystick device and control unit are mounted on a mobile robot and a control system according to this embodiment is mainly composed of a mobile robot will be described.

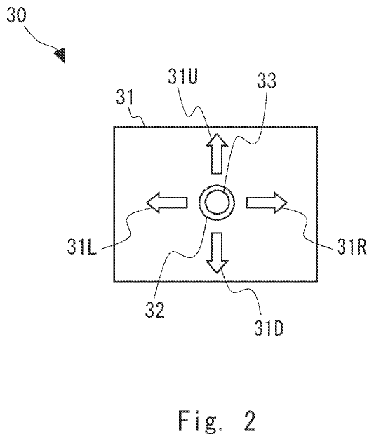

With reference to FIGS. 1 and 2, a mobile robot according to this configuration example will be described. FIG. 1 is a schematic perspective view showing one example of a mobile robot as a control system according to this embodiment and FIG. 2 is a top view showing one example of a joystick device mounted on the mobile robot shown in FIG. 1.

A mobile robot 1 shown in FIG. 1 is configured to be able to convey an object to be conveyed stored in a storage box to a destination indicated by a user terminal device (not shown), a host management apparatus (not shown) or the like and to convey the object to be conveyed according to a user operation accepted by the joystick device. However, another configuration in which the control system according to this embodiment does not include a user terminal device or a host management apparatus may instead be employed, and the aforementioned instruction in this case can be accepted from a display unit 16 and an operation interface 16a of the mobile robot 1 that will be described later. In this embodiment, a control method for causing the mobile robot 1 to perform an autonomous movement and a control method for causing the mobile robot 1 to move according to a user operation are not particularly limited, and the control that will be described below may be performed for the switching.

As shown in FIG. 1, the mobile robot 1 may include a body part 10, a carriage part 20, and a joystick device 30. In FIG. 1, the x-direction coincides with the forward/backward directions of the mobile robot 1, the y-direction coincides with the left/right directions of the mobile robot 1, and the z-direction coincides with the height direction of the mobile robot 1. As a matter of course, the mobile robot 1 shown in FIG. 1 is merely one example of the mobile robot according to this configuration example, and the shape, other functions and the like of the mobile robot 1 are not limited as long as the mobile robot 1 includes the joystick device and the control unit described above, and is configured in such a way that the modes of the mobile robot 1 can be switched.

The body part 10 is mounted on the carriage part 20. The body part 10 may schematically include a rectangular parallelepiped housing, and various components are disposed in the housing. Further, the body part 10 includes a door 15 and the aforementioned storage box that serves as a storage space hermetically closed by the door 15. The above-mentioned storage box includes, for example, multi-stage shelves, and the availability state of each stage can be managed. For example, the mobile robot 1 can update the availability state of each stage by disposing various sensors such as a weight sensor in each stage. Further, the door 15 may be configured so that it can be locked by an electric key or the like. When, for example, the mobile robot 1 arrives at the conveyance destination, a person who receives the conveyed object unlocks the door 15 by the electric key. Alternatively, when the mobile robot 1 arrives at the conveyance destination, the door 15 may be automatically unlocked.

Further, the body part 10 may include a control unit 11, which is an example of the aforementioned control unit, in a housing as a control box or the like. Hereinafter, an example in which the control unit 11 performs control on the carriage part 20 that performs autonomous movement as described above, specifically, control of a drive unit 21 that will be described later will be described with some examples.

The control unit 11 may be implemented, for example, by an integrated circuit, and may be implemented, for example, by a processor such as a micro processor unit (MPU) or a central processing unit (CPU), a working memory, a non-volatile storage apparatus or the like. This storage apparatus may store a control program executed by the processor and the processor may load this program into a working memory to execute the loaded program, thereby implementing the function of the control unit 11. The control unit 11 may be referred to as a control computer.

Cameras 12 which capture images ahead the mobile robot 1 in the traveling direction are disposed on the front surface of the body part 10. As shown in the drawing, the side of the mobile robot 1 on which the cameras 12 are disposed is defined as the front of the mobile robot 1. That is, when the mobile robot 1 is moving under normal circumstances, the forward direction of the mobile robot 1 is the traveling direction as indicated by an arrow. In this example, two cameras 12 function as stereo cameras. That is, the two cameras 12 having the same angle of view are horizontally arranged with an interval therebetween. The images captured by the cameras 12 are output as image data. Note that this image data may be still image data or may be moving image data. When the image data is still image data, still image data can be obtained for every image-capturing interval. It is possible to calculate the distance to the subject and the size thereof based on the image data of the two cameras 12. The control unit 11 can detect a person, an obstacle, or the like present ahead the mobile robot 1 in the moving direction by analyzing the images taken by the cameras 12. When there is a person, an obstacle, or the like ahead the mobile robot 1 in the traveling direction, the mobile robot 1 moves along the route while avoiding it. Further, when the control system is a system that includes a host management apparatus, the image data captured by the cameras 12 can be transmitted to the host management apparatus.

Further, the mobile robot 1 may include a range sensor group that measures a distance to a nearby object. As examples of the range sensor group, as shown in FIG. 1, a front/rear range sensor 13 and a left/right range sensor 14 may be provided on the exterior of the body part 10. The mobile robot 1 may measure a distance to the nearby object in the front/rear direction of the mobile robot 1 by the front/rear range sensor 13 and measure a distance to the nearby object in the left/right direction of the mobile robot 1 by the left/right range sensor 14.

For example, the front/rear range sensor 13 is disposed on each of the front and rear surfaces of the housing of the body part 10. The left/right range sensor 14 is disposed on each of the left-side and right-side surfaces of the housing of the body part 10. Each of the front/rear range sensor 13 and the left/right range sensor 14 is, for example, an ultrasonic range sensor or a laser range finder that detects the distance to the nearby object. When the distance to the nearby object detected by the front/rear range sensor 13 or the left/right range sensor 14 becomes equal to or shorter than a threshold distance, the mobile robot 1 may be controlled so that it decelerates or stops.

The mobile robot 1 may recognize a nearby object and/or determine its own position by analyzing image data output from the cameras 12 and detection signals output from the front/rear range sensor 13 and the left/right range sensor 14.

Further, the body part 10 of the mobile robot 1 may include a display unit 16 and an operation interface 16a disposed on the upper surface of the body part 10. The display unit 16 displays a Graphical User Interface (GUI) image, which is an operation image, which is a part of the operation interface 16a. The user touches the operation image displayed on the display unit 16 by a touch panel disposed on the display unit 16 is a part of the operation interface 16a, whereby the instruction input from the user can be accepted.

The display unit 16 is, for example, a liquid-crystal panel or an organic electro luminescence display, and displays the face of a character (e.g., a mascot) in an illustration and/or presents (i.e., shows) information about the mobile robot 1 in text or using an icon. It is possible, by displaying the face of the character on the display unit 16, to give people in the area around the mobile robot 1 an impression that the display unit 16 is as if the face of the robot. The display unit 16 and the like provided in the mobile robot 1 can be used as the user terminal device.

The carriage part 20 may schematically include a rectangular parallelepiped housing, and various components are disposed in the housing. For example, the drive unit 21 is housed in the carriage part 20. The drive unit 21 includes a driving wheel(s) 22 and a caster(s) 23. The driving wheel 22 is a wheel for moving the mobile robot 1 forward, backward, to the left, and to the right. The caster 23 is a driven wheel to which no driving force is supplied, and rolls so as to follow the driving wheel 22. The drive unit 21 includes a driving motor(s) (not shown) and drives the driving wheel(s) 22.

For example, the drive unit 21 supports, inside the housing, two driving wheels 22 and two casters 23, all of which are in contact with the surface on which the mobile robot travels. The two driving wheels 22 are arranged so that their rotational axes coincide with each other. Each of the driving wheels 22 is independently rotationally driven (i.e., independently rotated) by motors (not shown). The driving wheels 22 rotate according to control command values provided from the control unit 11. Each of the casters 23 is a trailing wheel, and is disposed in such a manner that its pivoting shaft, which vertically extends from the drive unit 21, rotatably supports the wheel at a point which is deviated from the rotating shaft of the wheel, and follows the driving wheel in the moving direction of the drive unit 21.

The mobile robot 1, for example, moves in a straight line when the two driving wheels 22 are rotated in the same direction at the same rotational speed, and turns around the vertical axis passing through substantially the center of the two driving wheels 22 when these wheels are rotated in the opposite direction at the same rotational speed. Further, the mobile robot 1 can move forward while turning left or right by rotating the two driving wheels 22 in the same direction at different rotational speeds. For example, the mobile robot 1 turns right by setting the rotational speed of the left driving wheel 22 higher than that of the right driving wheel 22. Conversely, the mobile robot 1 turns left by setting the rotational speed of the right driving wheel 22 higher than that of the left driving wheel 22. That is, the mobile robot 1 can move in a straight line, rotate on its own axis, or turn right or left in an arbitrary direction by individually controlling the rotational direction and the rotational speed of each of the two driving wheels 22.

The joystick device 30 may include a body part 31, a stick member 32, and a button 33. The joystick device 30 is mounted on the body part 10. However, it is sufficient that the joystick device 30 be disposed in a place where at least the stick member 32 and the button 33 can be operated by a user. A part or all of the body part 31 may be incorporated into the body part 10. For example, the part or all of the body part 31 may be embedded, in the body part 10.

The stick member 32 is a member for accepting a moving operation to forward, backward, left and right to move the mobile robot. The shape of the stick member 32 may be, for example, but not limited thereto, a bar-shaped member. This moving operation may indicate, for example, a moving operation to forward, backward, left and right. The moving operation will be referred to as a joystick operation.

The button 33 is a button that is disposed at the tip of the stick member 32 and accepts a pressing operation for switching an autonomous moving mode and a user operation mode. This pressing operation may also be referred to as an operation of switching the modes, or more simply a switching operation. The button 33, which is a member for accepting the pressing operation, may also be referred to as a button switch, a pushbutton switch or the like. The button 33 is not limited to a mechanical button but may be, for example, a touch-type button such as a capacitive button. The method for detecting the pressing operation and the shape of the button are not particularly limited. The button 33 may be of an automatic reset type. That is, when the button 33 is pressed, an output signal rises to ON, and when the button 33 is not pressed any longer, such as when the user releases his/her finger from the button 33, the output signal falls to OFF. However, this is merely an example. The button 33 may be, for example, a button whose output signal rises when it is pressed and falls when the rising ends, or may be a button that outputs a certain signal for a certain period of time between rising and falling. Further, while an example in which the button 33 is disposed at the tip of the stick member 32 will be described, the button 33 may instead be disposed on a side surface of the stick member 32, that is, on a surface gripped by the user.

The body part 31 may include, although not shown in the drawings, a detection unit that detects the joystick operation and the pressing operation by the stick member 32 and the button 33, respectively, and accepts the detected operations, and an interface or a communication unit that passes results of the detection in the detection unit to the control unit 11. Further, as illustrated in FIG. 2, arrows 31U, 31D, 31L, and 31R showing the operation directions of the stick member 32 to up/down (front/rear) and right/left may be shown on the surface of the body part 31. As a matter of course, the stick member 32 is not limited to accepting the joystick operation only in the four directions of up/down and right/left, that is, specifying the angle by the unit of 90 degrees, and may be able to accept a joystick operation specifying angles finer than 90 degrees. Further, the aforementioned detection unit may be configured to detect an inclination angle (elevated angle) of the stick member 32 even when the stick member 32 is tilted to a certain direction. In this case, the control unit 11 is able to perform control so as to increase the speed or the acceleration of the mobile robot 1 in accordance with the detected inclination angle.

In the autonomous moving mode, the mobile robot 1 is controlled to autonomously move. On the other hand, in the user operation mode, the mobile robot 1 is controlled to accept the joystick operation by the stick member 32 and to move in accordance with this joystick operation. In either mode, the pressing operation of the button 33 can be accepted, and the current mode is switched to the other mode due to this pressing operation. When the control system is a system that includes a host management apparatus, in the autonomous moving mode, the mobile robot 1 autonomously moves due to the control by the host management apparatus, whereas, in the user operation mode, the mobile robot 1 moves in accordance with the operation in the joystick device 30. Further, when the control system is a system including the host management apparatus, switching between these modes can be performed by the host management apparatus.

In this embodiment, at the time of switching between the autonomous moving mode and the user operation mode, the following control is performed. That is, when the mobile robot 1 is in the autonomous moving mode, the control unit 11 switches the mode to the user operation mode when falling of a press signal in response to the aforementioned pressing operation has been detected. Further, when the mobile robot 1 is in the user operation mode, the control unit 11 switches the mode to the autonomous moving mode when rising of a press signal in response to the aforementioned pressing operation has been detected.

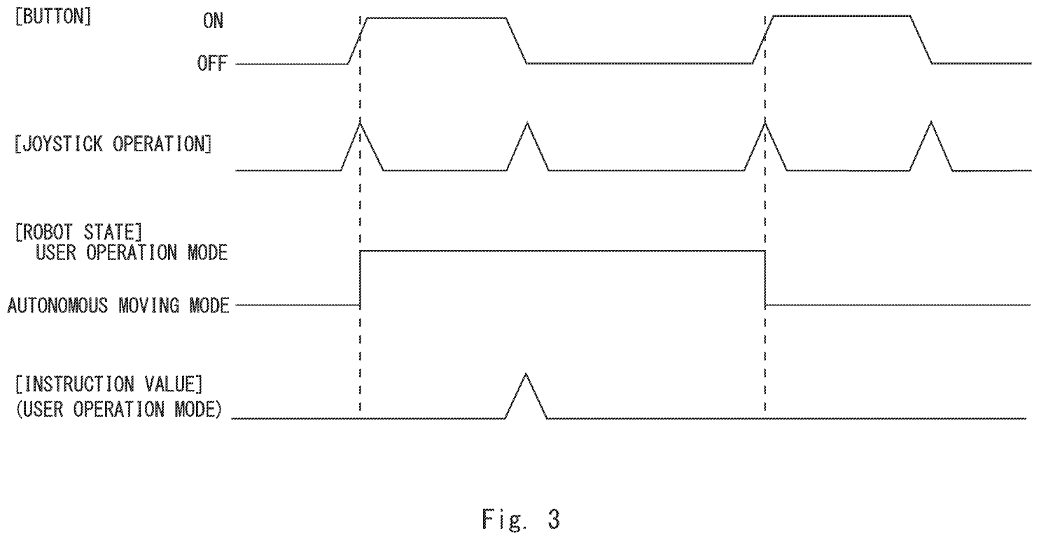

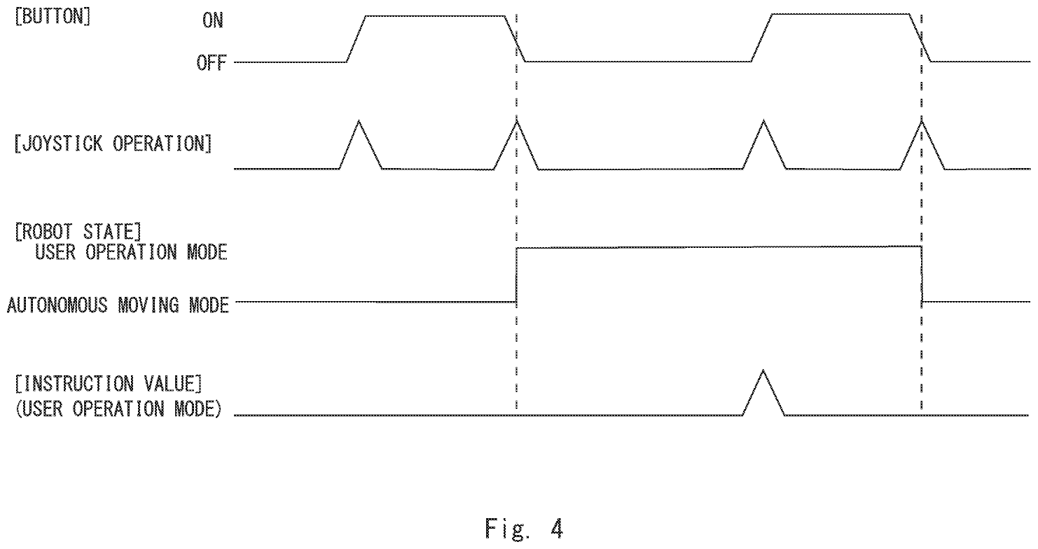

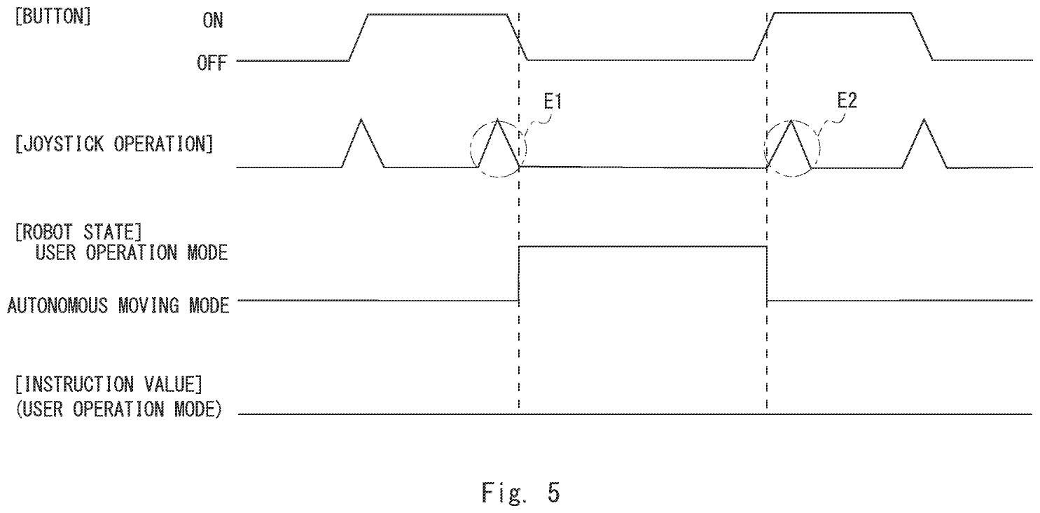

Next, with reference to FIGS. 3 to 5, the effectiveness of the aforementioned control will be described. FIG. 3 is a timing chart showing an example of response to operations by a button and a stick member in a mobile robot according to a first comparative example and FIG. 4 is a timing chart showing an example of response to operations by a button and a stick member in a mobile robot according to a second comparative example. FIG. 5 is a timing chart showing one example of response to operations by the button 33 and the stick member 32 in the mobile robot 1 shown in FIG. 1.

Both the first comparative example and the second comparative example include a mobile robot including a joystick device including a button and a stick member, like in the joystick device 30 that includes the button 33 and the stick member 32 described with reference to FIGS. 1 and 2. However, in both comparative examples, the control unit performs control that is different from that performed by the control unit 11 according to this embodiment.

FIG. 3 shows an example of response to an operation in the control unit according to the first comparative example (hereinafter, a first control unit). When the first control unit accepts an operation of pressing a button (ON operation), the first control unit performs control in such a way that the mobile robot is switched from the autonomous moving mode to the user operation mode at a rising edge of a press signal indicating the above pressing operation. However, the pressing operation may be performed by the user holding the stick member. At this time, an erroneous joystick operation may be accepted concurrently with the pressing operation, which may cause the mobile robot to move in an unintended direction. Specifically, when the erroneous joystick operation has been performed after the button is turned on and the rising edge of the press signal has been detected but before the button is turned on next time and the rising edge of the press signal is detected, a valid instruction value is input to the mobile robot as an instruction value in the user operation mode, which causes the first control unit to perform control to move the mobile robot in the direction indicated by the joystick operation, regardless of the user's intention.

FIG. 4 shows an example of response to an operation in the control unit according to the second comparative example (hereinafter, a second control unit). When the second control unit accepts an operation of pressing a button (ON operation), the second control unit performs control in such a way that the mobile robot is switched from the autonomous moving mode to the user operation mode at a falling edge of a press signal indicating the above pressing operation. However, the pressing operation may be performed by the user holding the stick member. At this time, an erroneous joystick operation may be accepted concurrently with the pressing operation, which may cause the mobile robot to move in an unintended direction. Specifically, when the erroneous joystick operation has been performed after the button is turned on and the falling edge of the press signal has been detected but before the button is turned on next time and the falling edge of the press signal is detected, a valid instruction value is input to the mobile robot as an instruction value in the user operation mode, which causes the second control unit to perform control to move the mobile robot in the direction indicated by the joystick operation, regardless of the user's intention.

FIG. 5 shows an example of response to the operation in the control unit 11. Unlike the aforementioned comparative examples, in the response to the operation shown in FIG. 5, even when the aforementioned erroneous joystick operation has been performed, the control unit 11 performs control assuming that there is no instruction value of this joystick operation or the instruction value is zero.

Specifically, as shown in FIG. 5, when the mobile robot 1 is in the autonomous moving mode, the control unit 11 switches the mode to the user operation mode when a falling edge of the press signal in response to the pressing operation of the button 33 has been detected. Accordingly, a joystick operation before a falling edge is detected (for example, a joystick operation shown by E1) is not reflected, since it is control in the autonomous moving mode. Further, as shown in FIG. 5, when the mobile robot 1 is in the user operation mode, the control unit 11 switches the mode to the autonomous moving mode when a rising edge of the press signal in response to the pressing operation of the button 33 has been detected. Accordingly, the joystick operation after the rising edge is detected (for example, a joystick operation shown by E2) is not reflected since it is control in the autonomous moving mode. Note that the timing of detecting the falling edge and the timing of detecting the rising edge may both be predetermined based on the performance and the like of the detection unit.

By performing the aforementioned control, in this embodiment, even when the user has erroneously performed a joystick operation concurrently with the pressing operation, it is possible to prevent the joystick operation from being reflected in the movement control of the mobile robot 1 for a period longer than that in the first and second comparative examples. Further, delay of a time until mode switching is reflected in response to the press signal can also be minimized in this embodiment.

As described above, in the control system according to this embodiment, it is possible to prevent the mobile robot 1 capable of switching between the autonomous moving mode and the user operation mode by a user performing an operation of switching these modes using the joystick device 30 from performing an unintended movement due to an unnecessary operation input by the user. Accordingly, according to this embodiment, it is possible to limit the unintended movement of the mobile robot due to the unnecessary input and to prevent occurrence of an event that endangers the surroundings. Further, for example, since there is no need to provide an emergency stop button or the like in the body part 10, it is possible to prevent an emergency stop button from being pushed by someone other than the user, for example, as a prank.

Further, as described above, when the mobile robot 1 is in the autonomous moving mode, the control unit 11 switches the mode to the user operation mode when falling of a press signal in response to the aforementioned pressing operation has been detected. Further, when the mobile robot 1 is in the user operation mode, the control unit 11 switches the mode to the autonomous moving mode when rising of a press signal in response to the aforementioned pressing operation has been detected. However, even in a case where the control unit 11 is configured to be able to perform only one of the above control operations, the effect achieved at the time of switching of one mode to the other mode may be achieved.

Further, the control unit 11 may perform control other than that described in the aforementioned control example. The control unit 11 may perform control for switching whether or not to accept a moving operation by the stick member 32 based on information indicating which one of the autonomous moving mode and the user operation mode the mobile robot is in and a change in the press signal in response to the operation of pressing the button 33. The information indicating which one of the aforementioned modes the mobile robot is in is information indicating the mode at the timing when the press signal is detected, and may be information at a timing when switching based on a press signal has not been performed. However, the information at the aforementioned timing can be obtained even with the information indicating the mode after the switching. Further, a change in the press signal in response to the operation of pressing the button 33 indicates the change in the switching signal in response to the operation of switching the modes by the button 33. Note that the control unit 11 may directly accept, when there is no switching signal and the mode is the user operation mode, the aforementioned moving operation signal and perform control to move the mobile robot 1 based on the aforementioned moving operation signal.

As described above, the control unit 11 can switch whether or not to employ a moving operation signal in response to the moving operation by the stick member 32, that is, whether or not to accept the moving operation signal based on a change in the aforementioned switching signal and the mode before the aforementioned switching signal is detected. Note that the change in the aforementioned switching signal may indicate the change itself in the aforementioned switching signal or may indicate a change in the signal indicated by results of predicting subsequent changes in the signal after the change in the signal. Further, it can be said that the control for switching whether or not to accept a moving operation signal is control for determining whether or not to switch the modes in response to the aforementioned switching signal or control for determining whether or not to accept or ignore the moving operation signal in response to the aforementioned switching signal.

For example, the control unit 11 determines whether or not a predetermined period of time has passed in a state in which the button 33 is being pushed after the aforementioned switching signal has started to rise, that is, after the button 33 is pushed. When the predetermined period of time has passed, the aforementioned moving operation signal can be accepted. In addition, this example of making the determination is an example where it is required to manage a time elapsed since a switching signal is detected, unlike the example in which rising or falling of the switching signal is detected.

The predetermined period of time in the user operation mode may be set shorter than the predetermined period of time in the autonomous moving mode. The reason therefor becomes similar to the reason stated above with regard to the example in which rising of the aforementioned switching signal is detected in the user operation mode and falling of the aforementioned switching signal is detected in the autonomous moving mode. Accordingly, a time during which the moving operation by the stick member 32 is not accepted in the user operation mode may be made shorter than that in the autonomous moving mode, whereby mode switching, that is, switching to the autonomous moving mode may be immediately performed. This is because, since the autonomous moving mode is a mode in which the moving operation by the stick member 32 is not accepted in the first place, an erroneous operation regarding the moving operation is not performed.

Further, due to a reason similar to that stated above, the aforementioned determination may be made, for example, only for the autonomous moving mode, and it may be determined whether or not a predetermined period of time has passed after the aforementioned switching signal has started to rise, that is, after the button 33 is pushed. In this case, the control unit 11 may accept the aforementioned moving operation signal only after a predetermined period of time has passed.

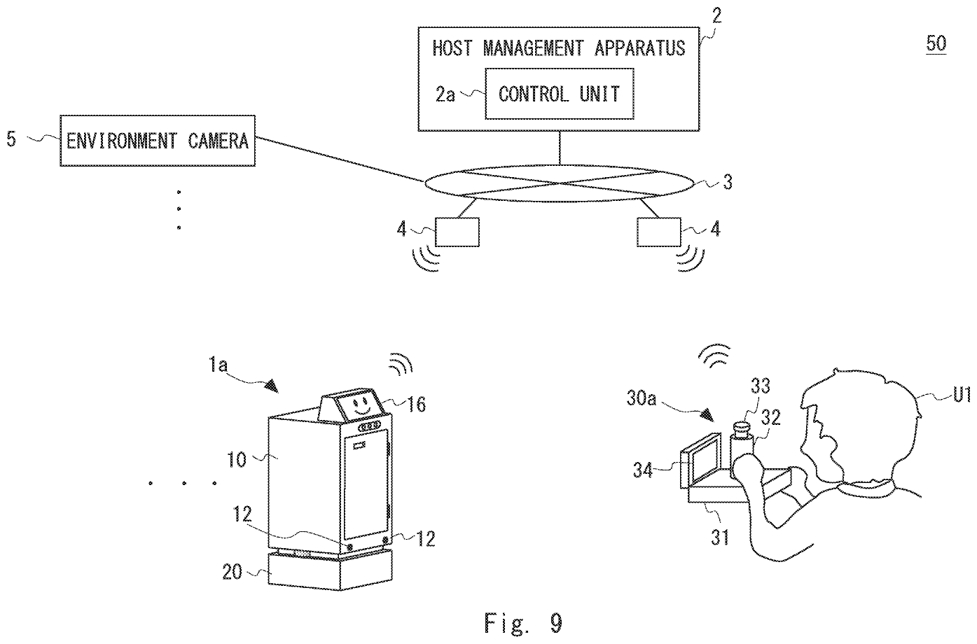

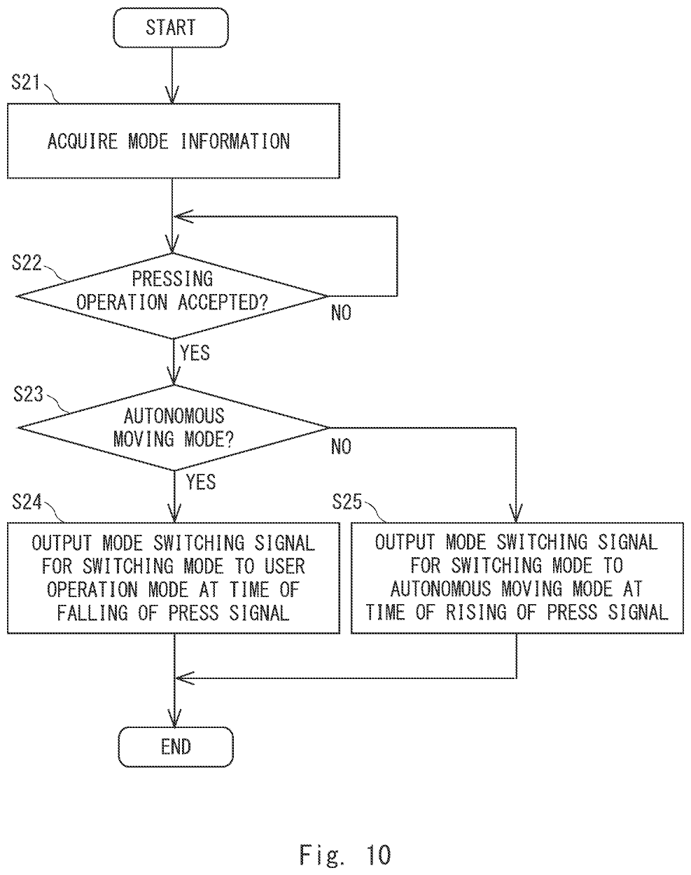

While the example in which the joystick device 30 is mounted on the mobile robot 1 has been described in FIG. 1, the joystick device 30 may be attached in such a way that it can be removed. In this case, if the joystick device 30 has been removed, control that will be described later with reference to FIGS. 9 and 10 may be performed. The communication between the joystick device 30 and the mobile robot 1 after the joystick device 30 is removed may be switched from wired communication to wireless communication or the communication between them may be originally wireless communication even before the joystick device 30 is removed.

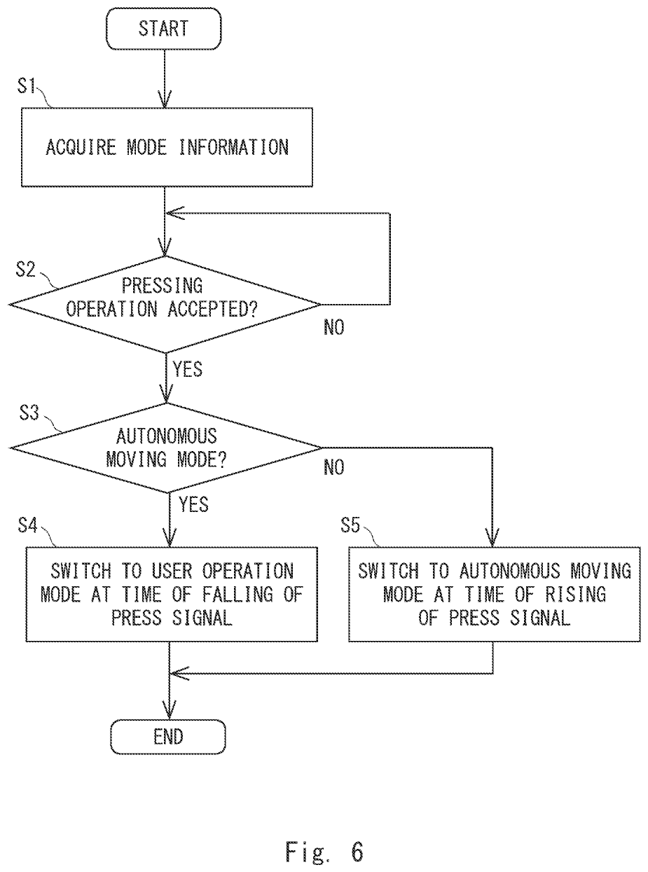

Next, with reference to FIG. 6, one example of the aforementioned control method in the mobile robot 1 will be described. FIG. 6 is a flowchart for describing one example of the control method in the mobile robot 1.

In this control method, first, the control unit 11 acquires mode information indicating which one of the modes the mobile robot 1 is in (Step S1). Next, the control unit 11 determines whether or not a pressing operation has been accepted (Step S2). When the pressing operation has not been accepted (NO), the control unit 11 waits until the pressing operation is accepted (until it is determined to be YES) while holding the mode information.

Next, the control unit 11 determines whether or not the mode information acquired in Step S1 indicates the autonomous moving mode (i.e., whether or not the mode information does not indicate the user operation mode) (Step S3). When it is determined to be YES, the control unit 11 performs control so that the mode is switched to the user operation mode at the time of falling of the press signal (Step S4), and ends the processing. On the other hand, when it is determined to be NO in Step S3, the control unit 11 performs control to switch the mode to the autonomous moving mode at the time of rising of the press signal (Step S5), and ends the processing. Note that the processing in Step S1 may be performed when it is determined to be YES in Step S2.

As described above, the control method according to this embodiment may include the following mode switching processing. This mode switching processing may include processing of switching the mode to the user operation mode at the time of falling of the press signal in response to the pressing operation when the mobile robot 1 is in the autonomous moving mode, and processing of switching the mode to the autonomous moving mode at the time of rising of the press signal in response to the pressing operation when the mobile robot 1 is in the user operation mode.

Note that the aforementioned program incorporated into the control unit 11 may be a program for causing a control computer, which is the control unit 11, or more specifically, a processor of the control unit 11 to execute the aforementioned mode switching processing. In the configuration in which the control system includes a host management apparatus, a program for causing the control computer, which is the control unit connected to the mobile robot 1 to execute the aforementioned mode switching processing may be incorporated into this control computer.

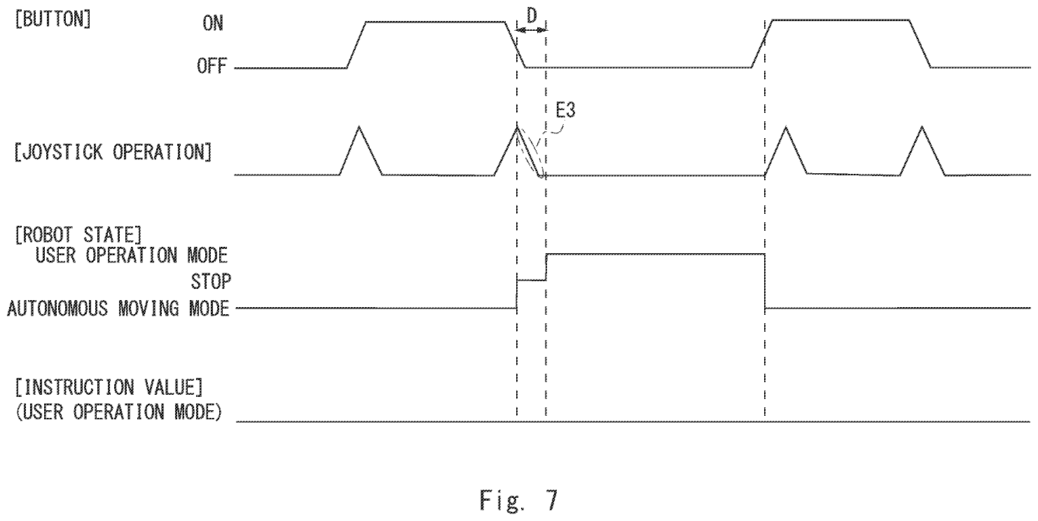

Referring next to FIG. 7, other control examples will be described. FIG. 7 is a timing chart showing another example of response to operations by the button 33 and the stick member 32 in the mobile robot 1.

In this control example, when the mobile robot 1 is in the autonomous moving mode, the control unit 11 stops or decelerates the mobile robot 1 at the time of falling of the press signal in response to the pressing operation, and switches the mode to the user operation mode after a predetermined period of time has elapsed.

Unlike the control example illustrated in FIG. 5, in this control example, as shown in FIG. 7 which shows an example of the response to the operation, even when an erroneous joystick operation has been performed, the influence of the joystick operation can be eliminated for a period during which the mobile robot 1 is stopped or decelerated.

For example, as shown in FIG. 7, when the mobile robot 1 is in the autonomous moving mode, the control unit 11 stops the mobile robot 1 at the time of detection of the falling edge of the press signal in response to the pressing operation of the button 33, and switches the mode to the user operation mode after a predetermined period of time D passes. Accordingly, even when a joystick operation is performed after the falling edge is detected, if this joystick operation is performed before the predetermined period of time D elapses (e.g., a joystick operation shown by E3 in FIG. 7), this joystick operation is not reflected since it is the control in the stop period. In this control example as well, control for switching the mode to the autonomous moving mode when the mobile robot 1 is in the user operation mode is similar to the control example illustrated in FIG. 5.

According to the above control, in this embodiment, it is possible to prevent an unintended movement of the mobile robot 1 due to an unnecessary input at the time of switching from the autonomous moving mode to the user operation mode for a period longer than that in the control example illustrated in FIG. 5 by the aforementioned predetermined period of time, that is, for a period longer than that in the control example illustrated in FIG. 5. At this time, by stopping the mobile robot 1, an unintended movement of the mobile robot 1 can be eliminated for a period longer than that in the control example illustrated in FIG. 5 by the aforementioned predetermined period of time. On the other hand, by decelerating the mobile robot 1, it is possible to eliminate or reduce the unintended movement of the mobile robot 1 by the aforementioned predetermined period of time without requiring power to move the mobile robot 1 again, i.e., in a low power consumption. In either case, it is possible to further reduce an unintended movement of the mobile robot due to an unnecessary input and prevent occurrence of an event that endangers the surroundings compared to that in the control example illustrated in FIG. 5.

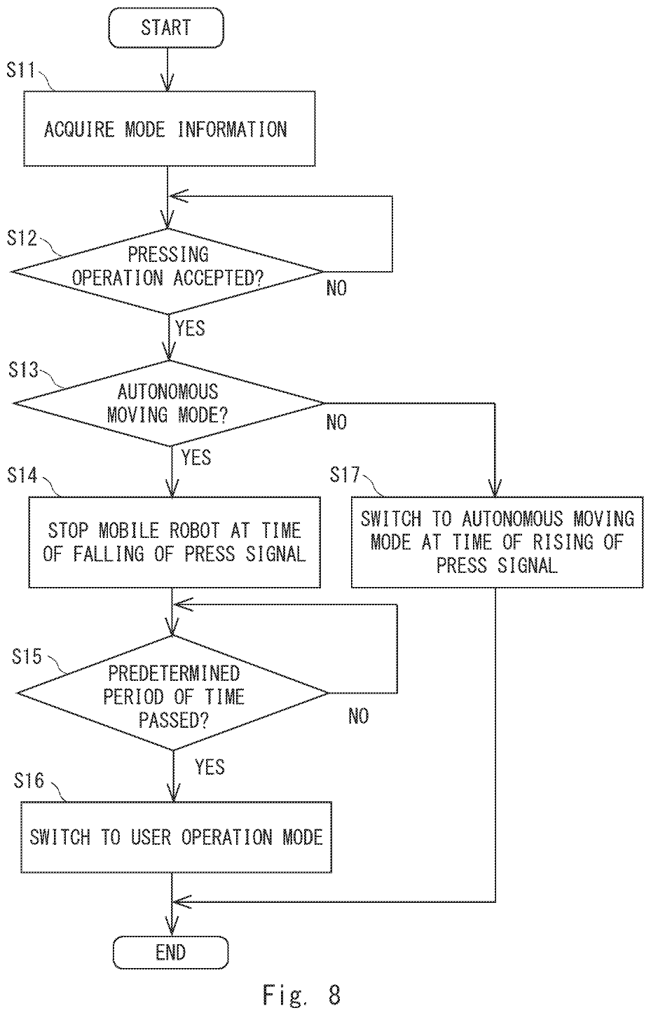

Next, with reference to FIG. 8, an example of another control method in the mobile robot 1 will be described. FIG. 8 is a flowchart for describing another example of the control method in the mobile robot 1.

In this control method, processing similar to that in Steps S1-S3 and S5 in the control method shown in FIG. 6 is performed (Steps S11-S13 and S17, respectively). When it is determined to be YES in Step S13, the control unit 11 may stop the mobile robot 1 at the time of falling of the press signal (Step S14). The mobile robot 1 may be decelerated instead of being stopped. Then, the control unit 11 determines whether or not a predetermined period of time has passed (Step S15). After the predetermined period of time has passed, the control unit 11 performs control so that the mode is switched to the user operation mode (Step S16), and ends the processing. On the other hand, when it is determined to be NO in Step S13, the control unit 11 performs control so that the mode is switched to the autonomous moving mode at the time of rising of the press signal (Step S17), and ends the processing.

Note that the aforementioned program incorporated into a control computer, which is the control unit 11 or a control unit connected to the mobile robot 1 is a program including the processing as illustrated in Steps S14 and S15.

Next, with reference to FIGS. 9 and 10, a configuration example in which the joystick device 30 is not mounted on the mobile robot 1 and the joystick device performs an operation at a position spaced away from the mobile robot will be described in brief. While an example in which control of the autonomous movement of the mobile robot is performed by a host management apparatus will be described in the configuration example described below, the control of the autonomous movement may also be performed in the configuration example described with reference to FIG. 1 and the like as well.

FIG. 9 is a schematic diagram showing one configuration example of a conveyance system including the control system according to this embodiment. As shown in FIG. 9, a conveyance system 50 includes a mobile robot 1a having a configuration except for the joystick device 30 included in the mobile robot 1, a host management apparatus 2, a network 3, a communication unit 4, an environment camera 5, and a joystick device 30a. It is assumed that the conveyance system 50 is a system that performs conveyance by the mobile robot 1a and includes a control system in this configuration example. The conveyance system 50 may also include a user terminal device (not shown) such as a tablet computer or a smartphone for sending a request for conveying an object to the host management apparatus 2.

The mobile robot 1a and the joystick device 30a are connected to the host management apparatus 2 via a communication unit 4 and a network 3. The network 3 is a wired or wireless Local Area Network (LAN) or Wide Area Network (WAN). Further, the host management apparatus 2 and the environment camera 5 are connected to the network 3 by a wire or wirelessly. The communication unit 4 is, for example, a wireless LAN unit installed in the environment of its apparatus or the like. The communication unit 4 may be, for example, a general-purpose communication device such as a WiFi (registered trademark) router.

The host management apparatus 2, which is a management system that manages a plurality of mobile robots 1a, may include a control unit 2a that controls the host management apparatus 2. The control unit 2a may be implemented, for example, by an integrated circuit. The control unit 2a may be implemented, for example, by a processor such as an MPU or a CPU, a working memory, and a non-volatile storage apparatus. A control program executed by a processor may be stored in this storage apparatus and the processor may load this program into the working memory to execute the loaded program, whereby the function of the control unit 2a may be implemented. The control unit 2a may be referred to as a control computer.

The conveyance system 50 controls the mobile robot 1a in a predetermined facility and efficiently controls a plurality of mobile robots 1a while making the mobile robots 1a autonomously move in a certain facility. The facility may indicate various kinds of facility such as a medical and welfare facility, such as a hospital, a rehabilitation center, a nursing facility, and a facility in which aged persons live, a commercial facility, such as a hotel, a restaurant, an office building, an event venue, or a shopping mall, or other complex facilities.

In order to perform the above-described efficient control, a plurality of environment cameras 5 may be installed in a facility. The environment camera 5 acquires an image of a range in which people and the mobile robot 1a move, and outputs image data indicating this image. Note that this image data may be still image data or moving image data. When the image data is still image data, still image data is obtained for each image-capturing interval. Further, in the conveyance system 50, the host management apparatus 2 collects images acquired by the environment camera 5 and information based on the images. As the images used for controlling the mobile robot 1a, images and the like acquired by the environment camera 5 may be directly transmitted to the mobile robot 1a. In the user operation mode, the images used for controlling the mobile robot 1a may be transmitted to the joystick device 30a directly or via the host management apparatus 2. The environment camera 5 may be provided as a monitoring camera in a passage or a doorway in the facility.

The host management apparatus 2 may determine, for each conveyance task, a mobile robot 1a that will perform the conveyance task and transmit an operation command for causing the determined mobile robot 1a to perform the conveyance task to the mobile robot 1a. The mobile robot 1a may perform autonomous movement in such a way that the mobile robot 1a moves from the conveyance origin to the conveyance destination according to the operation command. The method for determining the conveyance route in this case is not particularly limited.

For example, the host management apparatus 2 assigns a conveyance task to a mobile robot 1a present at or near the conveyance origin. Alternatively, the host management apparatus 2 assigns the conveyance task to a mobile robot 1a which is moving toward the conveyance origin or the vicinity thereof. The mobile robot 1a to which the task is assigned moves to the conveyance origin to collect the object to be conveyed.

The joystick device 30a is a device for remotely operating the mobile robot 1a directly or via the host management apparatus 2. The joystick device 30a may include a communication function therefor and a display unit 34. The other configurations of the joystick device 30a are basically similar to those of the joystick device 30. In the configuration in which the conveyance system 50 manages a plurality of mobile robots 1a, it is assumed that the mobile robot 1a to be remotely controlled can be selected by the joystick device 30a in the user operation mode.

As the aforementioned communication function, the joystick device 30a may include a reception unit and an output unit. The reception unit receives, from the host management apparatus 2 or the mobile robot 1a, a signal indicating whether the mobile robot 1a is in the autonomous moving mode or the user operation mode. When the mobile robot 1a is in the autonomous moving mode, the aforementioned output unit outputs, to the mobile robot 1a, a mode switching signal for switching the mode to the user operation mode at the time of detection of falling of a press signal in response to a pressing operation. Further, when the mobile robot 1a is in the user operation mode, the aforementioned output unit outputs, to the mobile robot 1a, a mode switching signal for switching the mode to the autonomous moving mode at the timing of detection of rising of a press signal in response to a pressing operation. The aforementioned output unit includes a transmission unit that transmits the aforementioned mode switching signal to the mobile robot 1a directly or via the host management apparatus 2. It is assumed that the reception unit and the transmission unit perform reception and transmission of the signals via wireless communication. Upon receiving the mode switching signal in accordance with the signal, the control unit in the mobile robot 1a switches modes between the autonomous moving mode and the user operation mode.

As a matter of course, in the user operation mode, the joystick device 30a transmits a signal indicating a joystick operation by the stick member 32 to the mobile robot 1a directly or via the host management apparatus 2, and the mobile robot 1a moves according to the above signal. Further, in this configuration example as well, the other control examples described with reference to FIGS. 7 and 8 can be applied. When the mobile robot 1a is stopped or decelerated, the joystick device 30a may output a control signal for stopping or decelerating the mobile robot 1a, and the mobile robot 1a may stop or decelerate in accordance with the above control signal.

The display unit 34 may display an image indicated by image data received from the cameras 12 in the mobile robot 1a and an image indicated by image data received from the environment camera 5 which is installed near the mobile robot 1a. Accordingly, the user U1 can perform a joystick operation using the stick member 32 and a pressing operation for switching modes of the mobile robot 1a using the button 33.

Next, with reference to FIG. 10, an example of the aforementioned control method in the mobile robot 1a will be described. FIG. 10 is a flowchart for describing one example of a control method of the mobile robot 1a in the conveyance system 50.

The above-described control method includes a method for controlling the joystick device 30a, including the following mode switching signal output processing. In this mode switching signal output processing, first, the control unit of the joystick device 30a acquires, by reception, a signal indicating which one of the modes the mobile robot 1a is in (a signal indicating mode information) from the mobile robot 1a (Step S21). Next, this control unit determines whether or not the pressing operation has been accepted (Step S22). When the pressing operation has not been accepted (NO), the control unit waits until the pressing operation is accepted (until it is determined to be YES) while holding the mode information.

Next, this control unit determines whether or not the mode information acquired in Step S21 indicates the autonomous moving mode (i.e., whether or not the mode information does not indicate a user operation mode) (Step S23). When it is determined to be YES, a mode switching signal for switching the mode to the user operation mode at the time of falling of a press signal is output to the mobile robot 1a (Step S24), and the processing is ended. On the other hand, when it is determined to be NO in Step S23, the mode switching signal for switching the mode to the autonomous moving mode at the time of rising of the press signal is output to the mobile robot 1a (Step S25), and the processing is ended. The processing in Step S21 may be performed at the timing when it is determined to be YES in Step S22.

As described above, the control method according to this embodiment may include the following mode switching signal output processing executed by the joystick device 30a. This mode switching signal output processing receives a signal indicating whether the mobile robot 1a is in the autonomous moving mode or the user operation mode. Further, when the mobile robot 1a is in the autonomous moving mode, this mode switching signal output processing outputs a mode switching signal for switching the mode to the user operation mode to the mobile robot 1a at the time of falling of a press signal in response to a pressing operation. Further, in this mode switching signal output processing, when the mobile robot 1a is in the user operation mode, a mode switching signal for switching the mode to the autonomous moving mode is output to the mobile robot 1a at the time of rising of a press signal in response to a pressing operation. As a matter of course, these signals may be output to the mobile robot 1a directly or via the host management apparatus 2.

Note that the program incorporated into the control unit of the joystick device 30a may be a program for causing a control computer, which is the control unit, or more specifically, a processor of the control computer to execute the above-described mode switching signal output processing.

Further, the aforementioned output unit is not limited to the examples described with reference to FIGS. 9 and 10, and may perform the output as follows, like the control described with regard to the control unit 11. That is, this output unit may output a mode switching signal to the mobile robot 1a to switch whether or not to accept a moving operation by the stick member 32 based on the signal received by the aforementioned reception unit and the change in the switching signal in response to the switching operation.

As described above, the examples described with reference to FIGS. 9 and 10 may achieve effects similar to those obtained in the examples described with reference to FIGS. 1 to 8. The examples described with reference to FIGS. 9 and 10 further achieve an effect that it becomes possible to perform a remote operation by the joystick device 30a.

### Alternative Examples

Note that the present disclosure is not limited to the above-described embodiments and may be changed as appropriate without departing from the spirit of the present disclosure.

For example, in place of the joystick device 30 according to the above-described embodiments, a joystick device in which a user performs a moving operation by moving his/her hand in a desired direction with the upper part of the joystick device wrapped in his/her palm, and performs an operation of switching the modes by pressing the upper part of the joystick device may be applied.

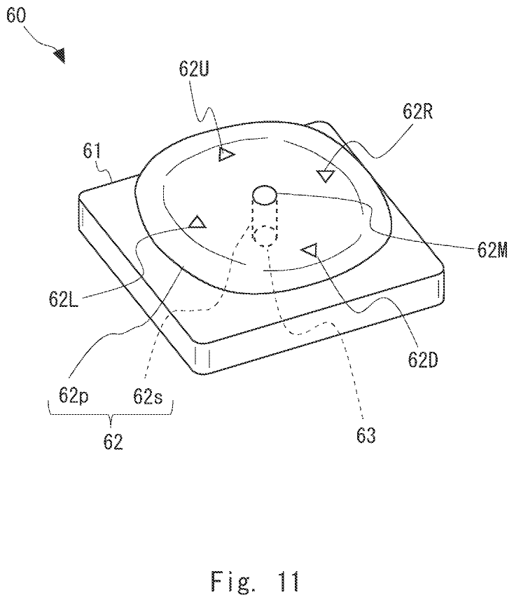

With reference to FIG. 11, one example of the aforementioned joystick device will be described. FIG. 11 is a perspective view showing one example of a joystick device that operates a mobile robot included in a control system according to an alternative example. A joystick device 60 shown in FIG. 11 may include a body part 61, a stick member 62, and a button 63. The stick member 62 may be a member including a stick part 62p on which a placement surface 62s is provided, the placement surface 62s being a surface on which the person's palm is placed, and a moving operation is assigned to the stick member 62. As illustrated in FIG. 11, arrows 62U, 62D, 62L, and 62R indicating the operation directions of the stick part 62p up/down (front/rear) and right/left may be shown or a mark 62M indicating the presence or the meaning of the button 63 may be shown in the placement surface 62s. Further, the button 63 may be disposed on a lower side of the stick part 62p in the body part 61 and may be pressed by a user who presses the placement surface 62s from above. The operation of switching the modes is assigned to this button 63.

In the above assignment as well, in a case where a user performs an operation of switching the modes using the joystick device 60, an unnecessary operation input may occur during this operation. Accordingly, the control described in the above-described embodiments may be performed for the joystick device 60 as well, whereby it is possible to prevent the mobile robot from performing an unintended movement due to the aforementioned unnecessary operation input.

Further, while the joystick device that accepts the moving operation and the switching operation by the same hand has been described, a joystick device that accepts the moving operation and the switching operation by different hands or an operation device of another operation form may instead be applied. Note that the joystick device and the operation device of another operation form may be called a controller.

Specifically, as the operation device included in the control system, an operation device in which a moving operation, which is an operation indicating a moving direction or a moving direction and a moving amount, and a switching operation for switching modes are associated as operations different from each other may be applied. An operation device in which the moving operation and the switching operation are associated as operations different from each other indicates an operation device in which the moving operation and the switching operation are assigned as different operations. In this case, the operation device includes a movement operation unit configured to accept a moving operation for moving the mobile robot and a switching operation unit for accepting a switching operation for switching modes between the autonomous moving mode and the user operation mode. When the operations are assigned as stated above, an unnecessary operation input may occur when a user performs an operation of switching the modes using the operation device. Therefore, the control described in the above-described embodiments is performed.

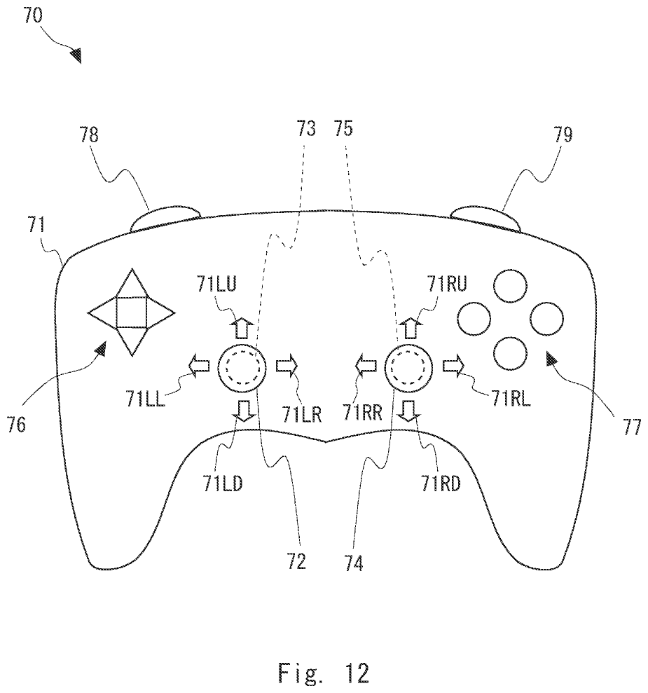

With reference to FIG. 12, an example of the joystick device that accepts the moving operation and the switching operation by different hands will be described. FIG. 12 is a top view showing one example of an operation device that operates the mobile robot included in the control system according to an alternative example. An operation device 70 shown in FIG. 12 may include a body part 71, stick members 72 and 74, buttons 73 and 75, a cross key 76, selection buttons 77, a left-side button 78, and a right-side button 79. Further, as illustrated in FIG. 12, arrows 71LU, 71LD, 71LL, and 71LR and arrows 71RU, 71RD, 71RL, and 71RR indicating the operation directions of up/down (front/rear) and right/left may be shown, for the stick member 72 and the stick member 74, respectively, on the surface of the body part 71. Further, the buttons 73 and 75 may be respectively disposed below the stick members 72 and 74 in the body part 71. These buttons may be the ones that are pushed as the user presses the stick members 72 and 74 from above.

Then, in the operation device 70, for example, one of the direction operation by the stick member 72, the direction operation by the stick member 74, the cross key 76, and the buttons 77 may be assigned to the moving operation. Further, in the operation device 70, a member different from the members assigned to the moving operation may be assigned to the operation of switching the modes. For example, in the operation device 70, one of the button 73, the button 75, and the cross key 76, one of the selection buttons 77, and one of the left-side button 78 and the right-side button 79 may be assigned to the operation of switching the modes.

In the aforementioned assignment example, since the right hand and the left hand are coupled to each other via the operation device 70, an unnecessary operation input may occur when the user performs an operation of switching the modes using the operation device 70. Accordingly, control described in the above-described embodiments may be performed for the operation device 70 as well, whereby it is possible to prevent the mobile robot from performing an unintended movement due to the aforementioned unnecessary operation input.

Further, in the control system, an operation device that accepts an operation using software may be applied to both the operation device that accepts the moving operation and the switching operation by the same hand, and the operation device that accepts the moving operation and the switching operation by different hands. The operation device that accepts an operation using software may be an operation device including a display device and a graphical user interface image displayed on the display device so that the image to be operated can be selected or moved. This graphical user interface image may include, for example, an image such as an icon for a moving operation that corresponds to the aforementioned movement operation unit and an image such as an icon for a switching operation that corresponds to the aforementioned switching operation unit.

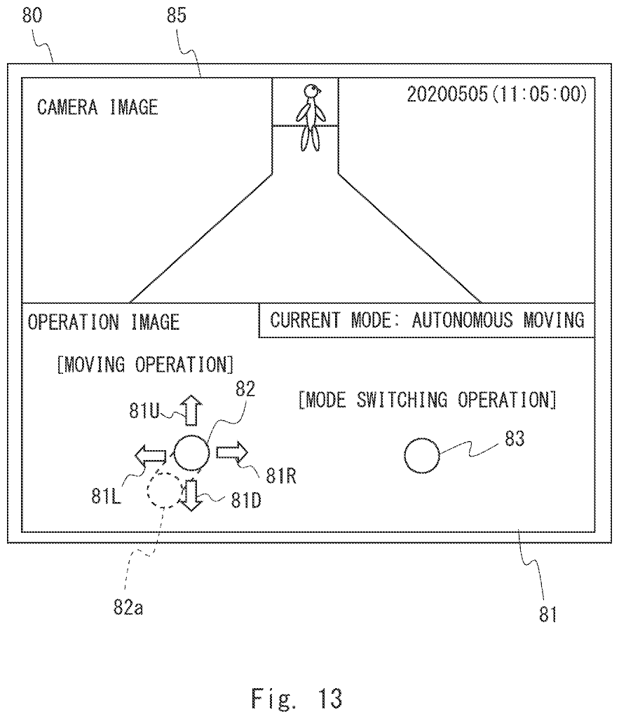

With reference to FIG. 13, one example of the operation device will be described. FIG. 13 is a top view showing another example of an operation device that operates the mobile robot included in the control system according to the alternative example. An operation device 80 shown in FIG. 13 is an operation device in which a graphical user interface image is displayed on a display device, and this image may include an operation image 81 and a camera image 85. As a matter of course, the camera image 85 may not be displayed when the remote operation is not taken into account. The operation image 81 may include a stick member image 82 for the moving operation and a button image 83 for the operation of switching the modes.

The button 83 is a button for accepting the switching operation, and information indicating the current mode may also be included in the operation image 81. Further, the stick member image 82 may be moved up/down, right/left or the like in a state in which it is being touched. In this case, as shown in FIG. 13, the original stick member image 82 may be kept displayed and a stick member image 82a, which is an image after the stick member image 82 is moved, may be displayed as well along with the image connecting them. Further, the operation image 81 may also include arrow images 81U, 81D, 81L, and 81R indicating the operation direction of the stick member image 82 up/down (front/rear) and right/left. In place of the stick member image 82, the images 81U, 81D, 81L, and 81R may serve as buttons for accepting the moving operations upward, downward, leftward, or rightward, respectively.

In the aforementioned assignment example, when the user performs an operation of switching the modes using the operation device 80, an unnecessary operation input may occur during the above operation. Accordingly, the control described in the above-described embodiments may be performed for the operation device 80 as well, whereby it is possible to prevent the mobile robot from performing an unintended movement due to the aforementioned unnecessary operation input.

Note that both the joystick device 60 and the operation devices 70 and 80 may be mounted in a state in which they are fixed to the mobile robot or may be attached to the mobile robot in such a way that they can be removed from the mobile robot, like in the examples of the joystick device 30 and the joystick device 30a.

Further, while the description has been made based on the assumption that the moving operation and the switching operation are accepted by the same hand or hands different from each other in the above-described embodiments and various alternative examples, this is merely an example. The above-described embodiments and the various alternative examples may also be applied, for example, to an operation device that accepts the moving operation and the switching operation by different fingers of one hand or an operation device that accepts the moving operation and the switching operation by feet different from each other.

Further, the above-described embodiments and various alternative examples have been described based on the assumption that the switching operation is the one for switching the mode between the autonomous moving mode and the user operation mode. However, this user operation mode may also include a plurality of modes regarding the response speed including a mode in which the mobile robot responds to the user operation at a high response speed or a mode in which the mobile robot responds to the user operation at a low response speed. Since an unnecessary operation input may occur also when the user performs an operation for switching these response speeds, by performing the control described in the above-described embodiments, it is possible to prevent the mobile robot from moving at an unintended response speed due to the aforementioned unnecessary operation input.

Further, the user operation mode in which the mobile robot moves based on a user operation is not particularly limited as long as the degree of involvement of the user operation is relatively higher than that in the autonomous moving mode in which the mobile robot autonomously moves. That is, the user operation mode is not limited to a mode in which all the movements of the mobile robot are performed based on the user operation and autonomous control by the mobile robot is completely excluded. Likewise, the autonomous moving mode is not limited to a mode in which full autonomous control by the mobile robot is performed and user operations are not accepted at all. The user operation mode and the autonomous moving mode may include, for example, the following first to third examples of modes.

In the first example, in the autonomous moving mode, the mobile robot performs autonomous traveling and determines to stop and start traveling while the user does not perform any operation, whereas in the user operation mode, the mobile robot performs autonomous traveling while the user performs the operation of stopping and starting traveling. In the second example, in the autonomous moving mode, the mobile robot performs autonomous traveling while the user performs the operation of stopping and starting traveling, whereas in the user operation mode, the mobile robot does not perform autonomous traveling while the user performs not only the operation of stopping and starting traveling but also the traveling operation. In the third example, in the autonomous moving mode, the mobile robot performs autonomous traveling and determines to stop and start traveling while the user does not perform any operation, whereas in the user operation mode, the mobile robot performs autonomous traveling regarding adjustment of the speed and avoidance of a collision while the user performs an operation such as a change in the traveling direction or a change in the route.

Further, the operation device such as the aforementioned joystick device may be used as a remote controller for operating a flying object or a control lever. That is, the control system according to this embodiment may also be applied to a flying object that can autonomously move as the mobile robot. Further, the control system according to this embodiment may be applied not only to a flying object but also various forms of mobile robots as the mobile robot.

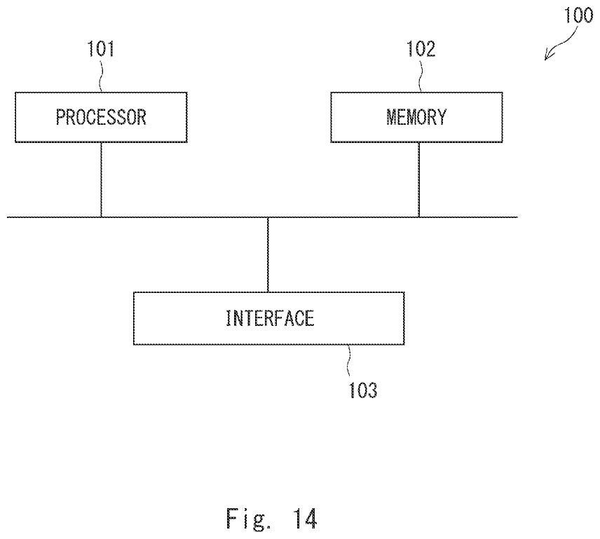

Further, each of the apparatuses included in the control system or the conveyance system 50 included in the above-described embodiments or various alternative examples may include, for example, the following hardware configuration. FIG. 14 is a diagram showing one example of a hardware configuration of the apparatus.

An apparatus 100 shown in FIG. 14 may include a processor 101, a memory 102, and an interface 103. The interface 103 may include, for example, a communication interface or interfaces with, for example, a drive unit, a sensor, an input/output device, and the like, which are required according to the apparatus.

The processor 101 may be, for example, a CPU, a Graphics Processing Unit (GPU), a Micro Processor Unit (MPU) also called a microprocessor. The processor 101 may include a plurality of processors. The memory 102 is composed of, for example, a combination of a volatile memory and a nonvolatile memory. The function of each apparatus is implemented by having the processor 101 load a program stored in the memory 102 and executing the loaded program while exchanging necessary information through the interface 103.

The aforementioned various programs include instructions (or software codes) that, when loaded into a computer, cause the computer to perform one or more of the functions described in the embodiments. The program may be stored in a non-transitory computer readable medium or a tangible storage medium. By way of example, and not a limitation, computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray (registered trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A control system for controlling a mobile robot capable of switching between an autonomous moving mode in which the mobile robot autonomously moves and a user operation mode in which the mobile robot moves based on a user operation, the control system comprising:
an operation device comprising a movement operation unit configured to accept a moving operation for moving the mobile robot and a switching operation unit configured to accept a switching operation for switching the modes between the autonomous moving mode and the user operation mode; and
a control unit configured to switch, based on information indicating which one of the autonomous moving mode and the user operation mode the mobile robot is in and a change in a switching signal in response to the switching operation, whether or not to accept the moving operation by the movement operation unit.

2. The control system according to claim 1, wherein, when the mobile robot is in the autonomous moving mode, the control unit switches the mode to the user operation mode when falling of the switching signal in response to the switching operation has been detected.

3. The control system according to claim 1, wherein, when the mobile robot is in the autonomous moving mode, the control unit stops or decelerates the mobile robot when falling of the switching signal in response to the switching operation has been detected, and switches the mode to the user operation mode after a predetermined period of time has elapsed.

4. The control system according to claim 1, wherein, when the mobile robot is in the user operation mode, the control unit switches the mode to the autonomous moving mode when rising of the switching signal in response to the switching operation has been detected.

5. The control system according to claim 1, wherein the operation device is a joystick device comprising a stick member and a button disposed in the stick member, the stick member being the movement operation unit and the button being the switching operation unit.

6. A control method for controlling a mobile robot capable of switching between an autonomous moving mode in which the mobile robot autonomously moves and a user operation mode in which the mobile robot moves based on a user operation using an operation device, wherein
the operation device comprises a movement operation unit configured to accept a moving operation for moving the mobile robot and a switching operation unit configured to accept a switching operation for switching the modes between the autonomous moving mode and the user operation mode, and
the control method switches whether or not to accept the moving operation by the movement operation unit based on information indicating which one of the autonomous moving mode and the user operation mode the mobile robot is in and a change in a switching signal in response to the switching operation.

7. The control method according to claim 6, comprising, when the mobile robot is in the autonomous moving mode, switching the mode to the user operation mode when falling of the switching signal in response to the switching operation has been detected.

8. The control method according to claim 6, comprising, when the mobile robot is in the autonomous moving mode, stopping or decelerating the mobile robot when falling of the switching signal in response to the switching operation has been detected, and switching the mode to the user operation mode after a predetermined period of time has elapsed.

9. The control method according to claim 6, comprising, when the mobile robot is in the user operation mode, switching the mode to the autonomous moving mode when rising of the switching signal in response to the switching operation has been detected.

10. The control method according to claim 6, wherein the operation device is a joystick device including a stick member and a button disposed in the stick member, the stick member being the movement operation unit and the button being the switching operation unit.

11. A non-transitory computer readable medium storing a program for controlling a mobile robot capable of switching between an autonomous moving mode in which the mobile robot autonomously moves and a user operation mode in which the mobile robot moves based on a user operation using an operation device, wherein
the operation device comprises a movement operation unit configured to accept a moving operation for moving the mobile robot and a switching operation unit configured to accept a switching operation for switching the modes between the autonomous moving mode and the user operation mode,
the program is a program for causing a control computer included in the mobile robot or connected to the mobile robot to execute mode switching processing, and
the mode switching processing is processing for switching whether or not to accept the moving operation by the movement operation unit based on information indicating which one of the autonomous moving mode and the user operation mode the mobile robot is in and a change in a switching signal in response to the switching operation.

12. The non-transitory computer readable medium according to claim 11, wherein the mode switching processing comprises processing for switching, when the mobile robot is in the autonomous moving mode, the mode to the user operation mode when falling of the switching signal in response to the switching operation has been detected.

13. The non-transitory computer readable medium according to claim 11, wherein the mode switching processing comprises processing for stopping or decelerating, when the mobile robot is in the autonomous moving mode, the mobile robot when falling of the switching signal in response to the switching operation has been detected, and switching the mode to the user operation mode after a predetermined period of time has elapsed.

14. The non-transitory computer readable medium according to claim 11, wherein the mode switching processing comprises processing for switching, when the mobile robot is in the user operation mode, the mode to the autonomous moving mode when rising of the switching signal in response to the switching operation has been detected.

15. The non-transitory computer readable medium according to claim 11, wherein the operation device is a joystick device comprising a stick member and a button disposed in the stick member, the stick member being the movement operation unit and the button being the switching operation unit.
